(19) United States
(12) Patent Application Publication
(10) Pub. No.: US 2004/0186473 A1
(43) Pub. Date: Sep. 23, 2004

(54) Spinal fixation devices of improved strength and rigidity

(76) Inventors: John R. Cournoyer, Norfolk, MA (US); Michael S. Varieur, Portsmouth, RI (US); P. Alan Ruiz, Mansfield, MA (US); Anthony R. Carlone, Bristol, RI (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/393,575

(22) Filed: Mar. 21, 2003

(51) Int. Cl. A61B 17/56

(52) U.S. Cl. 606/61

(57) ABSTRACT

This invention relates to spinal implants of improved strength and rigidity. One embodiment relates to U-shaped, top loading spinal rod receivers wherein the threading on the receiver ends above the top of the rod when the rod is in the locked positions. Other embodiments relate to improved thread designs based on minimizing the gap between the minor diameter of the receiver and the minor diameter of the securing member and by ensuring an equal or substantially equal number of full width threads on each of the flanges of the U-shaped receiver.

### BACKGROUND OF THE INVENTION

The present invention relates generally to spinal fixation devices of improved strength and rigidity.

Common spinal fixation techniques involve immobilizing the spine by using orthopedic rods, commonly referred to as spine rods, that run generally parallel to the spine. Spinal fixation is typically accomplished by exposing the spine posteriorly or anteriorly and fastening bone screws or hooks (“bone fasteners”) to the pedicles or laminae of the appropriate vertebrae. These bone fasteners may be of the polyaxial (e.g., as described in U.S. Pat. No. 5,672,176 (Biedermann) or U.S. Pat. No. 6,485,491 (Farris)) or monoaxial (e.g., as described in U.S. Pat. No. 5,738,658 (Halm) or U.S. Pat. No. 5,725,527 (Biedermann)) types. Receiving elements adapted for receiving a spine rod therethrough are then used to join the spine rods to the screws, hooks, extenders, and/or connectors. The aligning influence of the rods forces the spine to conform to a more desirable shape. In certain instances, the spine rods may be bent to achieve the desired curvature of the spinal column.

In coupling the rod to the bone fasteners, a problem arises when using top load, U-shaped receivers having a single securing member. The problem with these devices is that the upright legs or flanges of the receiver can experience splaying after or during implantation. During spine surgery, when the bone fasteners have already been implanted and a spinal rod has been introduced into the receiving element of the fixation device, insertion instruments are used to apply the securing screw to the receiver of the pedicle screws to contain the spinal rod. A light torque is generally used to first capture the spinal rod. Additional torque may be applied to the securing screw if compression and/or distraction are required. Once the surgeon is satisfied with the placement of the spinal rod, the recommended final tightening torque will be applied to the securing screw to secure the spinal rod in place.

Single securing screw implants will slip or fail if the design torque is exceeded. As the securing screw is inserted into the implant via screw threading, the screw eventually contacts the rod with a downward force for securing position of the implant and rod with respect to each other. As the fastener is turned, any extra space between the bottom of the rod and the implant is removed. As additional torque is applied, reactionary forces are generated within the fastener due to contact with the rod. The force on the rod is then counteracted by opposing forces acting on the threads of the implant and the fastener. At this moment, the interacting forces then cause deflection of the thread teeth on both the receiver and the securing screw which generates a slip angle between the interacting teeth. As this slip angle increases, force is created on the flanges of the implant causing the flanges to deflect outward away from the concentric centers of the receiver, securing screw, and rod. If the material of the flange is not strong enough to minimize the deflection of the flanges under these conditions, the deflection that occurs will allow the thread teeth of the securing screw to slip off the thread teeth of the flange. The resultant deformation of the flanges of the implant is then referred as head splay.

To prevent splaying, prior medical devices have included a nut, cap, clamp or similar apparatus to surround and hold the flanges of the fixation element together. For example, in U.S. Pat. No. 5,672,176 to Biedermann et al., a rod is placed into a slot in the fixation element, the locking member is engaged with the fixation element to press down via an intermediary part on the rod, and an outer nut is threaded on the outside of the fixation element.

There is therefore a need remaining in the industry for medical devices, and particularly orthopedic devices, which minimizes or prevents splaying of spinal fixation elements.

### SUMMARY OF THE INVENTION

This invention relates spinal implants of improved strength and rigidity.

Various embodiments relate to U-shaped, top loading spinal rod receivers wherein the threading on the receiver ends above the top of the rod when the rod is in the locked position. Other embodiments relate to improved thread designs based on minimizing the gap between the minor diameter of the receiver and the minor diameter of the securing member and by ensuring an equal or substantially equal number of full width threads on each of the flanges of the U-shaped receiver.

One embodiment of this invention relates to a bone anchor assembly comprising:

a. a bone fastener having an upper end and a lower end, a head at the upper end, and an anchoring element between the upper and lower ends;

b. a U-shaped receiver having an upper end and a lower end and having two flanges at its upper end, said receiver adapted to receive a stabilizing rod, a bore extending through the lower end of said receiver for receiving said fastener, threading beginning at said upper end and ending above the area of said receiver not occupied by said rod when said rod is in a locked position; and

c. a securing member having threads to engage the threading of said receiver to apply a force upon said rod positioned in said receiver and wherein said rod directly or indirectly contacts and applies a force upon said head for forcing said head against said lower end of the receiver for preventing further pivotal and rotational movement of said fastener and said receiver relative to one another.

Another embodiment relates to a bone screw comprising:

a. bone fastening end having threads to engage a bone; and

b. a U-shaped receiver end, said receiver having an upper end and a lower end and having two flanges at its upper end, said receiver adapted to receive a stabilizing rod, threading beginning at said upper end and ending above the area of said receiver not occupied by said rod when said rod is in a locked position; and

c. a securing member having threads to engage the threading of said receiver to apply a force upon said rod positioned in said receiver and wherein said rod contacts and applies a force upon said lower end of the receiver for preventing movement of said rod and said receiver relative to one another.

Yet other embodiment relates to a bone hook comprising:

a. a bone engaging end having a hook to engage a bone; and

b. a U-shaped receiver end, said receiver having an upper end and a lower end and having two flanges at its upper end, said receiver adapted to receive a stabilizing rod, threading beginning at said upper end and ending above the area of said receiver not occupied by said rod when said rod is in a locked position; and

c. a securing member having threads to engage the threading of said receiver to apply a force upon said rod positioned in said receiver and wherein said rod contacts and applies a force upon said lower end of the receiver for preventing movement of said rod and said receiver relative to one another.

A further embodiment relates to a bone anchor assembly comprising:

a. a bone fastener having an upper end and a lower end, a head at the upper end, and an anchoring element between the upper and lower ends thereof;

b. a U-shaped receiver having an upper end and a lower end and having two flanges at its upper end, said receiver adapted to receive a stabilizing rod, a bore extending through the lower end of said receiver for receiving said fastener, threading beginning at said upper end and ending in the area of said receiver above or below the area occupied by the top of said rod when said rod is in a locked position;

c. a securing member having threads to engage the threading of said receiver to apply a force upon said rod positioned in said receiver and wherein said rod directly or indirectly contacts and applies a force upon said head for forcing said head against said lower end of the receiver for preventing further pivotal and rotational movement of said fastener and said receiver relative to one another; and

d. wherein the number of full width threads of the threading on each of the U-shaped member flanges are equal or substantially equal.

Another embodiment relates to a bone screw comprising:

a. a bone fastening end having threads to engage a bone; and

b. a U-shaped receiver end, said receiver having an upper end and a lower end and having two flanges at its upper end, said receiver adapted to receive a stabilizing rod, threading beginning at said upper end and ending either above or below the area of said receiver not occupied by said rod when said rod is in a locked position;

c. a securing member having threads to engage the threading of said receiver to apply a force upon said rod positioned in said receiver and wherein said rod contacts and applies a force upon said lower end of the receiver for preventing movement of said rod and said receiver relative to one another; and

d. wherein the number of full width threads on the threading on each of the U-shaped flanges are equal or substantially equal.

Another embodiment relates a bone hook comprising:

a. a bone engaging end having a hook to a bone; and

b. a U-shaped receiver end, said receiver having an upper end and a lower end and having two flanges at its upper end, said receiver adapted to receive a stabilizing rod, threading beginning at said upper end and ending above or below the area of said receiver not occupied by said rod when said rod is in a locked position;

c. a securing member having threads to engage the threading of said receiver to apply a force upon said rod positioned in said receiver and wherein said rod contacts and applies a force upon said lower end of the receiver for preventing movement of said rod and said receiver relative to one another; and

d. wherein the number of full width threads of the threading on each of the U-shaped member flanges are equal or substantially equal.

Yet a further embodiment relates to a spinal implant comprising:

a. a U-shaped receiver end, said receiver having an upper end and a lower end and having two flanges at its upper end, said receiver adapted to receive a stabilizing rod, threading beginning at said upper end and ending above the area of said receiver not occupied by said rod when said rod is in a locked position;

b. a securing member having threads to engage the threading of said receiver to apply a force upon said rod positioned in said receiver and wherein said rod directly or indirectly contacts and applies a force upon said lower end of the receiver for preventing movement of said rod and said receiver relative to one another;

c. wherein the number of full width threads of the threading on each of the U-shaped member flanges are equal or substantially equal; and

d. wherein the spinal implant is selected from the group consisting of polyaxial and monoaxial types of screws, hooks, extenders, and connectors.

Finally, the concepts of centering and clocking (timing) as hereinafter disclosed, may be applied to methods of manufacturing spinal implants to eliminate or lessen splaying of the receiver.

### BRIEF DESCRIPTION OF THE DRAWINGS

FIG. 1 depicts a detailed view of the engagement of thread features between the U-shaped rod receiver and securing member relating to this invention.

FIG. 2 depicts top and orthogonal views of the U-shaped receiver aspect of this invention.

FIGS. 3a and 3b depict polyaxial screw embodiments of this invention.

FIGS. 4a and 4b depict polyaxial hook embodiments of this invention.

FIG. 5 depicts a monoaxial screw embodiment of this invention.

FIG. 6 depicts a monoaxial hook embodiment of this invention.

FIGS. 7a and 7b depict additional polyaxial screw embodiments of this invention.

FIGS. 8a and 8b depict additional polyaxial hook embodiments of this invention.

FIG. 9 depicts an additional monoaxial screw embodiment of this invention.

FIG. 10 depicts an additional monoaxial hook embodiment of this invention.

FIGS. 11a and 11b depict sacral extender embodiments of this invention.

### DETAILED DESCRIPTION OF PREFERRED EMBODIMENTS OF THE INVENTION

It is contemplated that the aspects of this invention hereinafter described relate to spinal implants of the open U-shaped receiver configuration which require a corresponding securing member. The securing member may be or a single set screw type (such as described in U.S. Pat. No. 5,005,562 (Cotrel)), a threaded cap type(such as described in U.S. Pat. No. 6,471,705 (Biedermann) or U.S. Pat. No. 6,440,137 (Horvath)), a set screw and ring combination (such as describes in U.S. Pat. No. 5,217,497 (Mehdian)), or a securing screw and outer nut combination (such as described in U.S. Pat. No. 5,725,527 (Biedermann)), the disclosures of which are hereby incorporated by reference. Thus where any threading type engagement is used with spinal implants of the open U-shaped receiver configuration, the benefits of this invention are equally applicable.

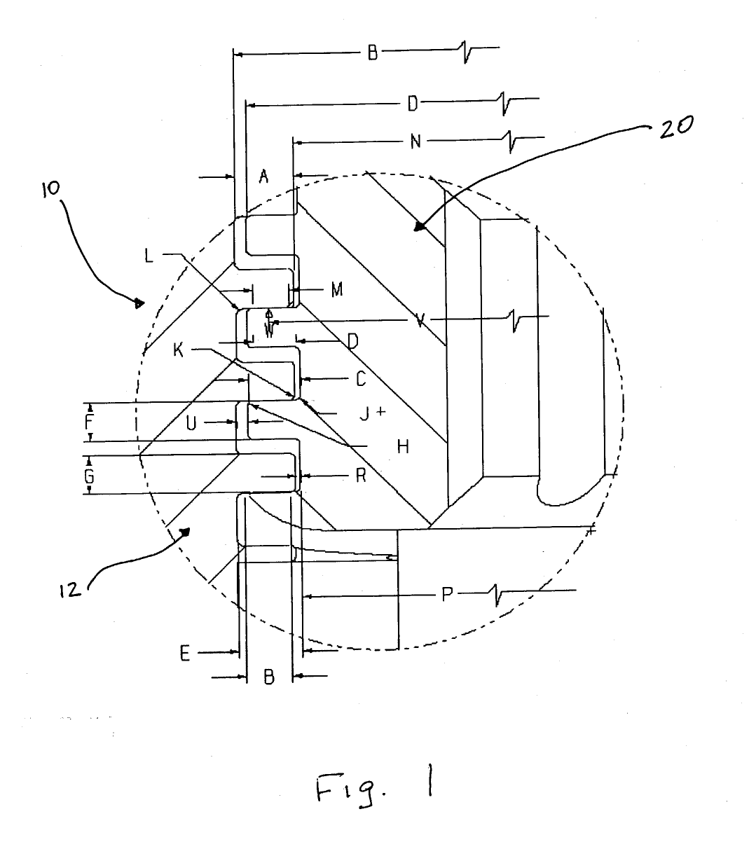

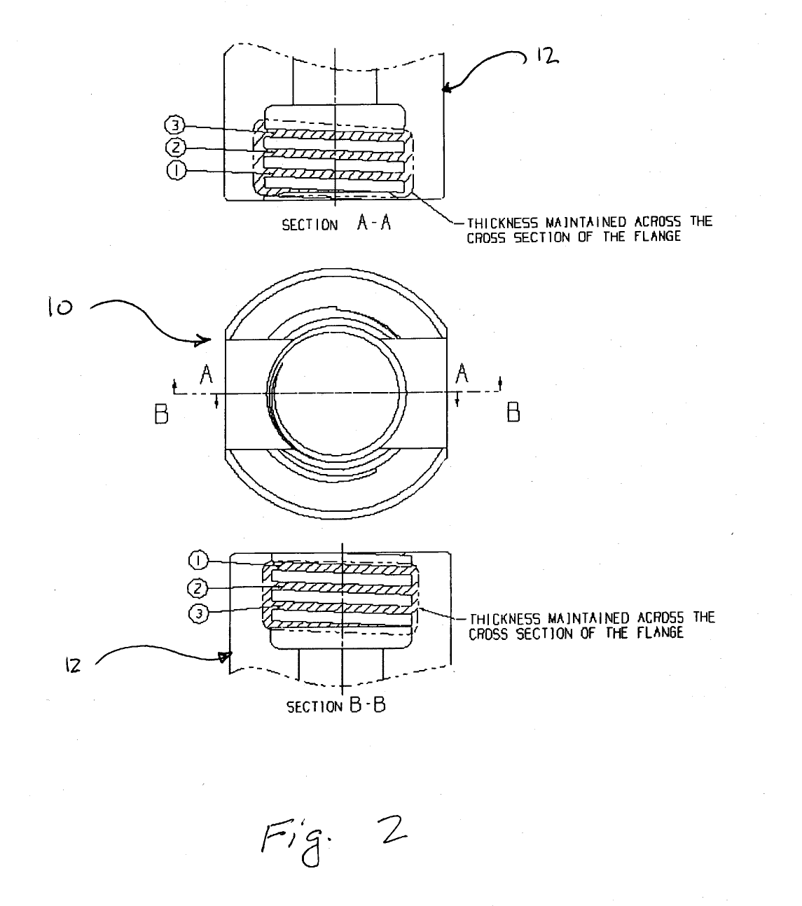

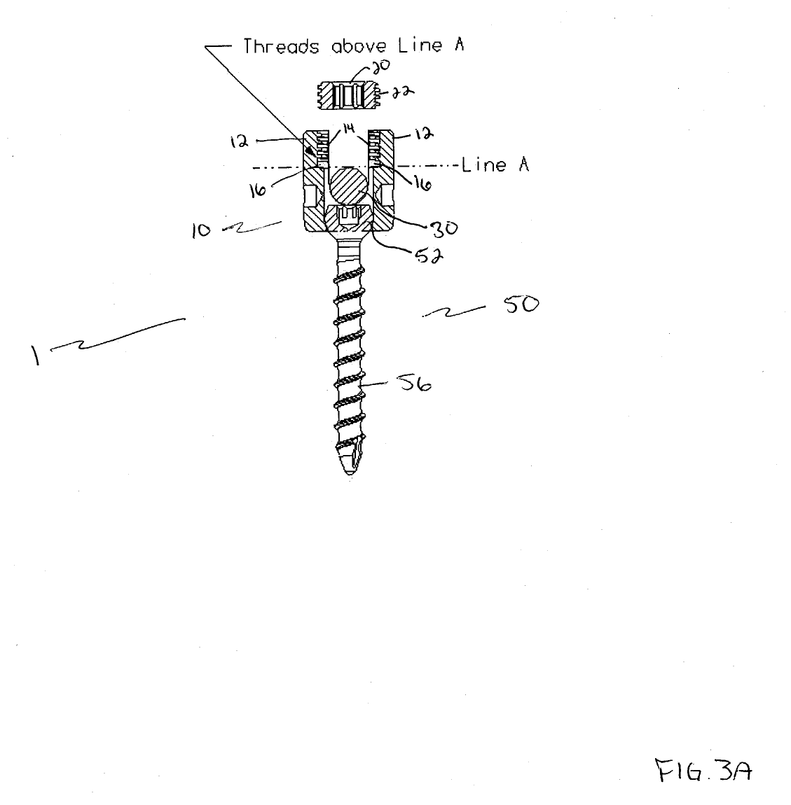

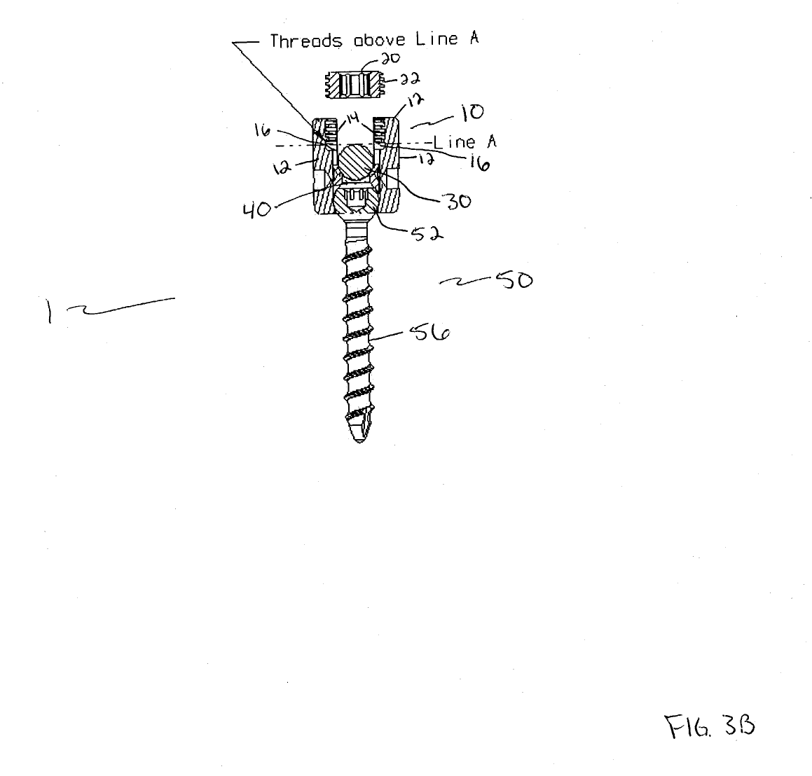

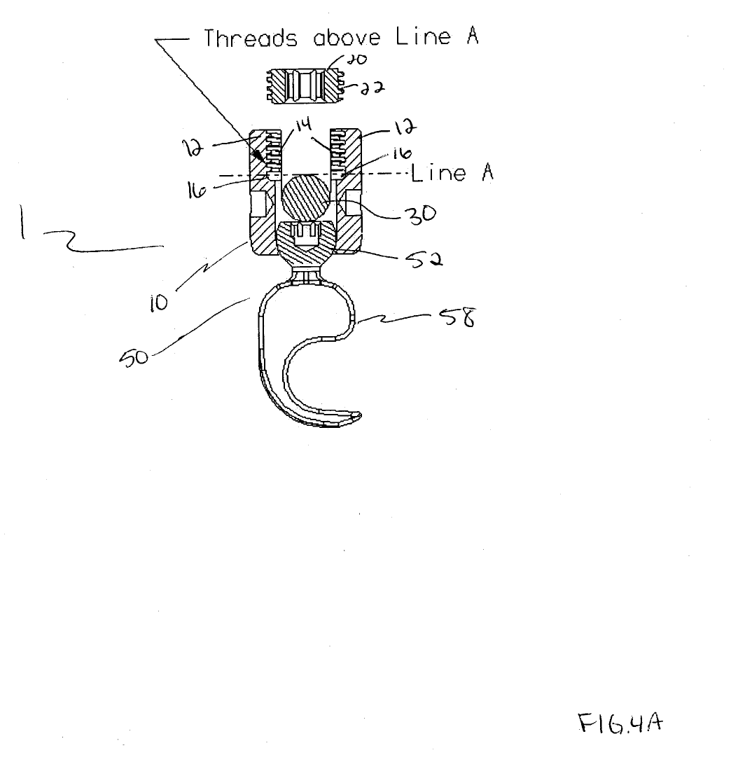

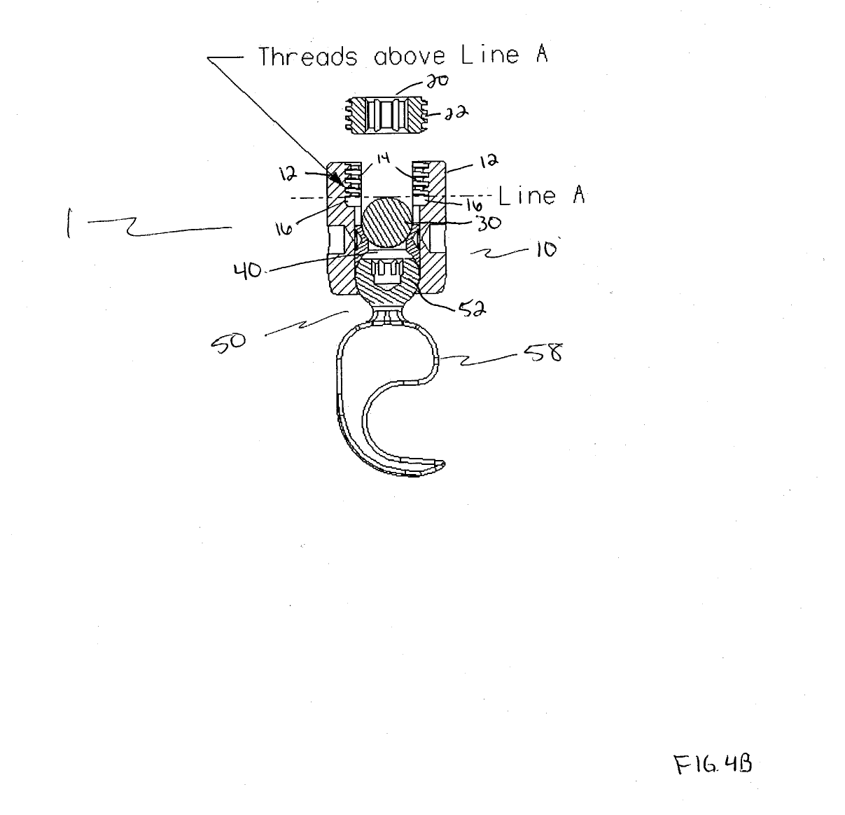

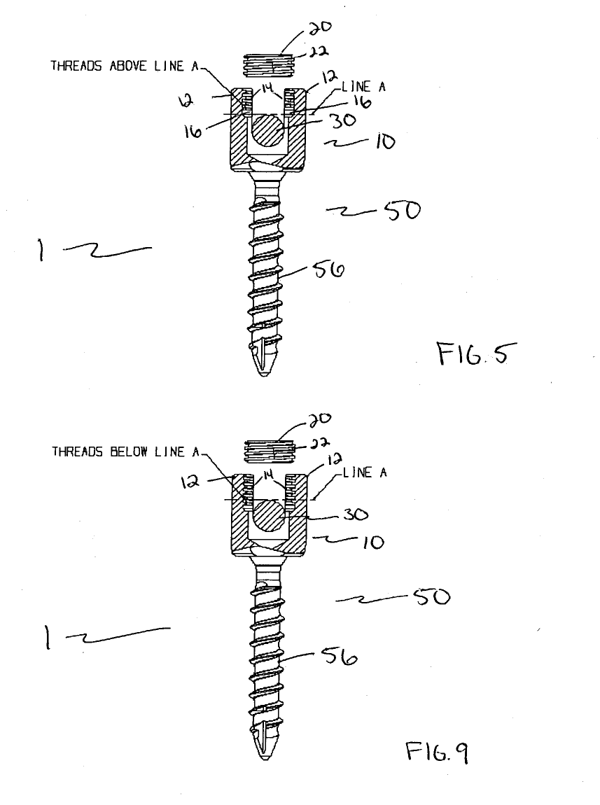

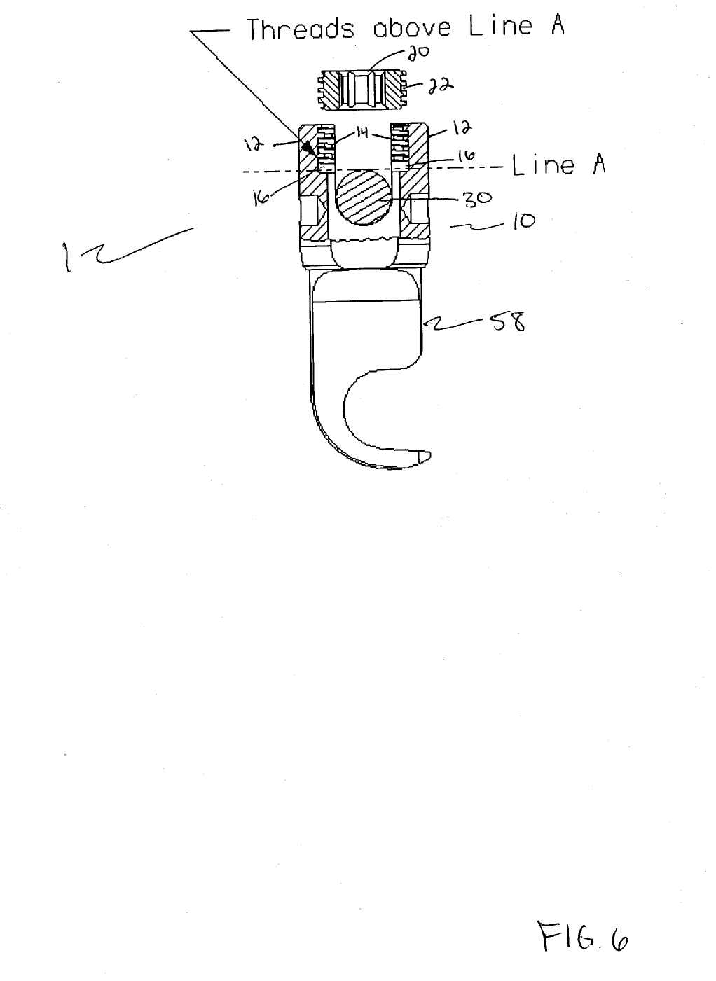

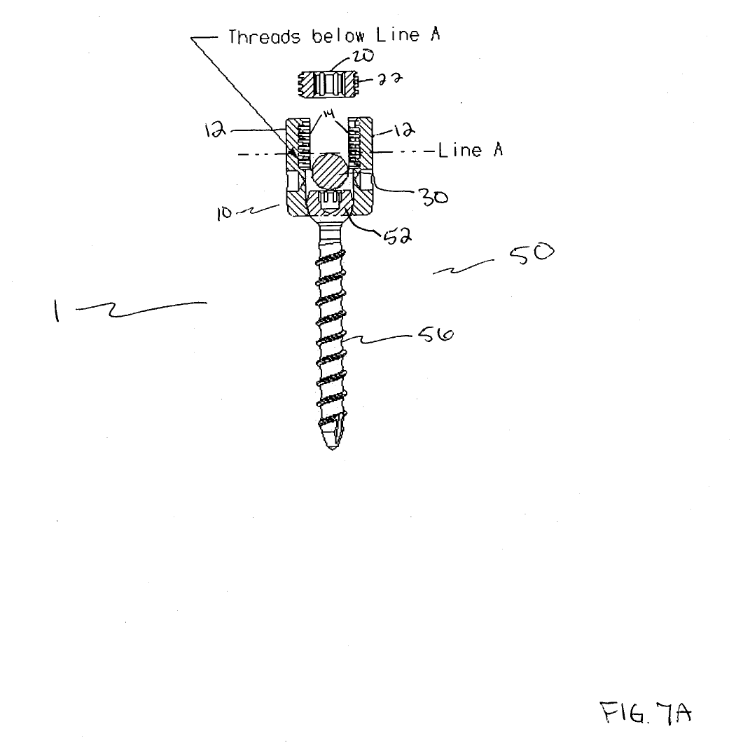

One aspect of this invention relates to the positioning of the centerline locations of a receiver or a spinal rod and its corresponding securing members, hereinafter referred to as centering. By way of non-limiting illustration, FIG. 1 depicts one type of engagement between receiver 10 and securing member 20. Table 1 summarizes the various dimensions depicted in FIG. 1.

TABLE 1

| | |
|---|---|
| A | Tooth length of the receiver tooth |
| B | Major diameter of receiver |
| C | Length of the securing member tooth |
| D | Major diameter of securing member |
| E | Distance between major diameter of receiver and the |
| | minor diameter of the securing member |
| F | Height of the securing member tooth |
| G | Height of the receiver tooth |
| H | Edge break of securing member tooth (2 Places) |
| J | Corner radii of the securing member tooth (2 Places) |
| K | Edge break of receiver tooth (2 Places) |
| L | Corner radii of the receiver tooth (2 Places) |
| M = E − R − | Length of contact between receiver and securing |
| U − H − K | member |
| N | Minor diameter of receiver |
| P | Minor diameter of securing member |
| R = (N − P)/2 | Distance between minor diameter of the receiver and the |
| | securing member |
| U = (B − D)/2 | Distance between the major diameters of receiver and |
| | securing member |
| V | Moment Arm |
| W | Force |

When the securing member 20 is inserted into the receiver 10 and contact with a rod is achieved, the force from the rod is transmitted to the threads of the securing member 20 and receiver 10. Referring to FIG. 1, which shows a single flange 12 of receiver 10, the force (W) creates a moment about the centerline of receiver 10. Symmetrically, there is also a moment created on the opposite flange of the assembly from the force acting on the opposite thread (not shown). These forces and moments are equal or substantially equal when the centerlines of receiver 10 and securing member 20 remain colinear.

During the tightening of securing member 20 onto the rod in receiver 10, securing member 20 can shift asymmetrically away from the center of receiver 10 towards one of the flanges 12 thereby resulting in an unequal distribution of force and hence, head splay. Thus by minimizing the distance (R) between the minor diameter of receiver 10 and the minor diameter of securing member 20, centering is achieved thereby limiting or minimizing splaying.

Another aspect of this invention relates to the correcting of another contributing factor leading to head splay is that the start point of the thread, also known as “timing”, is not controlled in conjunction with the start or “top” of the implant, therefore, the full-uninterrupted thread contact between the receiver and the securing member may vary over the opposite flanges. Thus in the machining of the U-shaped receiver threads are cut and the U-shaped channel is cut, ensuring that there are a equal or substantially equal number of full width threads (i.e., thread not run out, but maintaining its full thread width in the flange), the amount of force on each side of the flanges are equal or substantially equal, thus minimizing or eliminating splaying.

FIG. 2 provides an example of a view looking down upon receiver 10 with orthogonal views of flanges 12. As can be seen in this figure, there are equal or substantially equal number of threads of full thickness. These full thickness threads are identified by numbers \mathring{1}, \mathring{2}, and \mathring{3} of section views A-A and B-B.

Thus, the general concept of centering and timing are applicable (individually or in combination) to numerous types of spinal implants having top-loading, open U-shaped receivers including but not limited to screws, hooks, sacral extenders, cross connectors, lateral offset connectors, reduction screws, translation hooks, etc. of both the polyaxial and monoaxial types.

In other embodiments, the invention further relates to spinal implants having threads that end above the area of the receiver that is not occupied by the rod. It has been surprisingly found that implants of improved strength and rigidity are achieved. More specifically, we have found that head splaying is minimized by not continuing threading on the flanges below the top of the rod. It is believed that we have found that by having less threads, there is less of a tendency for the flange threads to impart a torque to splay the open end of the receiver during tightening as the securing member locks the rod into place. These other embodiments are described in the following drawings.

FIG. 3A shows spinal implant 1 having receiver 10 which comprises flanges 12 with threaded sections 14 ending above rod 30 and fastener portion 50 including head 52 and anchoring element in the form of polyaxial screw 56. Securing member has corresponding threads 22 to engage threads 14 of receiver 10 and to secure rod 30 within receiver 10. When securing member 20 is in its locked position, it applies force upon rod 30 which directly contacts and applies force upon head 52 forcing head 52 against the lower end of receiver 10 for preventing further pivotal and rotational movement of fastener 50 and receiver 10 relative to one another. Section 16 of receiver 10 does not contain any threads 14. Line A-A shows threads 14 ending above the top of rod 30.

FIG. 3B represents another embodiment for spinal implant 1 comprising receiver 10 having flanges 12 with threaded sections 14 ending above rod 30, intermediate member 40 and fastener portion 50 including head 52 and anchoring element in the form of polyaxial screw 56. Securing member 20 has corresponding threads 22 to engage threads 14 of receiver 10 and to secure rod 30 within receiver 10. When securing member 20 is in its locked position, it applies force upon rod 30 which directly contacts and applies force upon intermediate member 40 and indirectly applies force on head 52 forcing head 52 against the lower end of receiver 10 for preventing further pivotal and rotational movement of fastener 50 and receiver 10 relative to one another. Section 16 of receiver 10 does not contain any threads 14. Line A-A shows threads 14 ending above the top of rod 30.

FIG. 4A shows spinal implant 1 having receiver 10 which comprises flanges 12 with threaded sections 14 ending above rod 30 and fastener portion 50 including head 52 and anchoring element in the form of polyaxial hook 58. Securing member has corresponding threads 22 to engage threads 14 of receiver 10 and to secure rod 30 within receiver 10. When securing member 20 is in its locked position, it applies force upon rod 30 which directly contacts and applies force upon head 52 forcing head 52 against the lower end of receiver 10 for preventing further pivotal and rotational movement of fastener 50 and receiver 10 relative to one another. Section 16 of receiver 10 does not contain any threads 14. Line A-A shows threads 14 ending above the top of rod 30.

FIG. 4B represents another embodiment for spinal implant 1. Spinal implant 1 comprises receiver 10 having flanges 12 with threaded sections 14 ending above rod 30, intermediate member 40 and fastener portion 50 including head 52 and anchoring element in the form of polyaxial hook 58. Securing member 20 has corresponding threads 22 to engage threads 14 of receiver 10 and to secure rod 30 within receiver 10. When securing member 20 is in its locked position, it applies force upon rod 30 which directly contacts and applies force upon intermediate member 40 and indirectly applies force on head 52 forcing head 52 against the lower end of receiver 10 for preventing further pivotal and rotational movement of fastener 50 and receiver 10 relative to one another. Section 16 of receiver 10 does not contain any threads 14. Line A-A shows threads 14 ending above the top of rod 30.

FIG. 5 depicts another embodiment of spinal implant 1. This embodiment represents a monoaxial screw having receiver 10 having flanges 12 with threaded sections 14 ending above rod 30 and fastener portion 50 in the form of monoaxial screw 56. Securing member 20 has corresponding threads 22 to engage threads 14 of receiver 10 to apply a force upon rod 30 positioned within receiver 10 and rod 30 contacts and applies a force upon the lower end of receiver 10 for preventing movement of rod 30 and receiver 10 relative to each other. Section 16 of receiver 10 does not contain any threads 14. Line A-A shows threads 14 ending above the top of rod 30.

FIG. 6 depicts another embodiment of spinal implant 1. This embodiment represents a monoaxial hook comprising receiver 10 having flanges 12 with threaded sections 14 ending above rod 30 and fastener portion 50 in the form of hook 58. Securing member 20 has corresponding threads 22 to engage threads 14 of receiver 10 to apply a force upon rod 30 positioned within receiver 10 and rod 30 contacts and applies a force upon the lower end of receiver 10 for preventing movement of rod 30 and receiver 10 relative to each other. Section 16 of receiver 10 does not contain any threads 14. Line A-A shows threads 14 ending above the top of rod 30.

FIG. 7A shows another embodiment of spinal implant 1. This embodiment is a polyaxial screw comprising receiver 10 having flanges 12 with threaded sections 14 ending in the area of receiver 10 below the area occupied by top of rod 30. Line A-A depicts the location of the area defined by the top of rod 30. Spinal implant 1 further includes fastener portion 50 including head 52 and anchoring element in the form of polyaxial screw 56. Securing member 20 has corresponding threads 22 to engage threads 14 of receiver 10 and to secure rod 30 within receiver 10. When securing member 20 is in its locked position, it applies force upon rod 30 which directly contacts and applies force upon head 52 forcing head 52 against the lower end of receiver 10 for preventing further pivotal and rotational movement of fastener 50 and receiver 10 relative to one another.

FIG. 7B represents another embodiment of spinal implant 1 comprising receiver 10 having flanges 12 with threaded sections 14 ending in the area of receiver 10 below the area occupied by top of rod 30. Line A-A depicts the location of the area defined by the top of rod 30. Spinal implant 1 further comprises intermediate member 40 and fastener portion 50 including head 52 and anchoring element in the form of polyaxial screw 56. Securing member 20 has corresponding threads 22 to engage threads 14 of receiver 10 and to secure rod 30 within receiver 10. When securing member 20 is in its locked position, it applies force upon rod 30 which directly contacts and applies force upon intermediate member 40 and indirectly applies force on head 52 forcing head 52 against the lower end of receiver 10 for preventing further pivotal and rotational movement of fastener 50 and receiver 10 relative to one another.

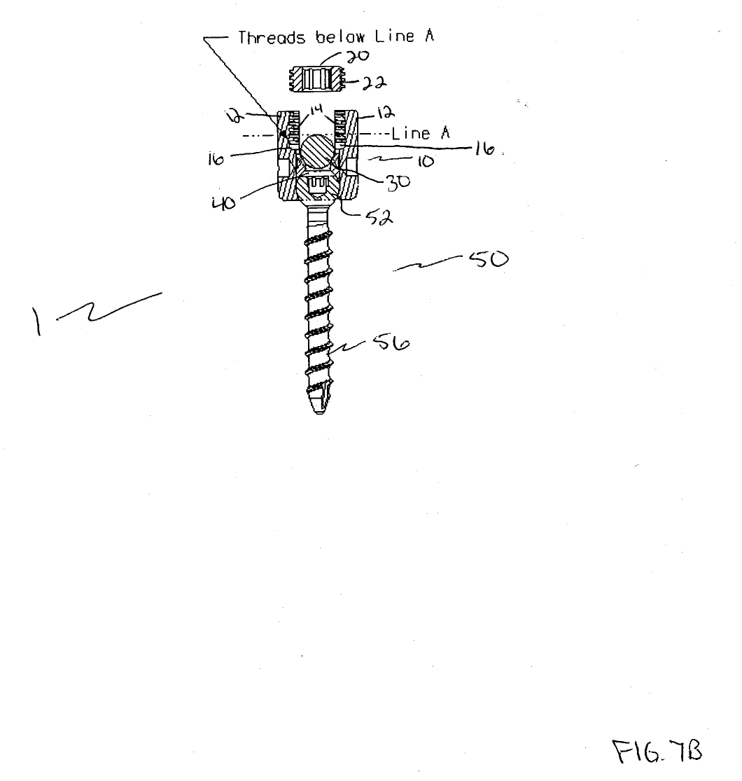

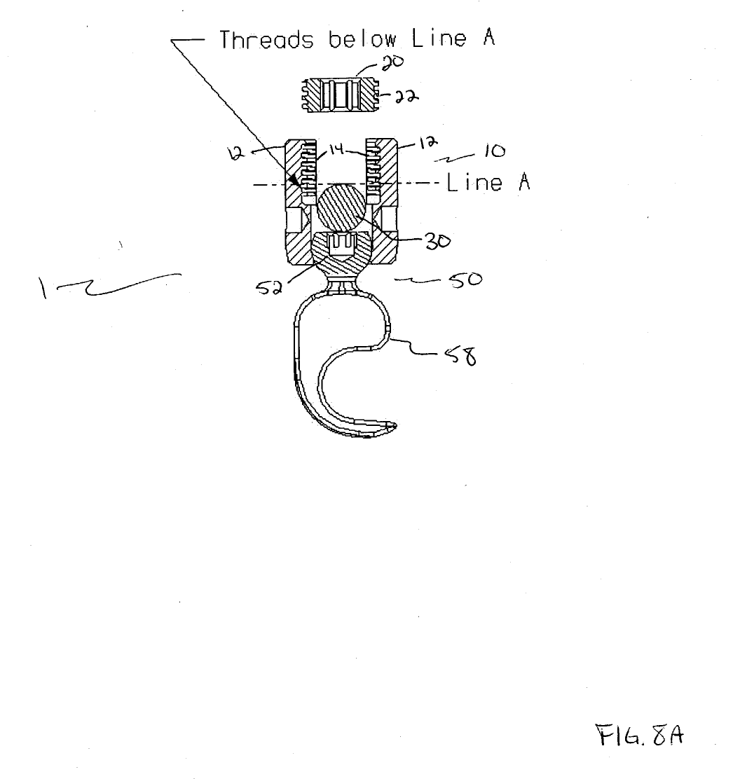

FIG. 8A shows spinal implant 1 having receiver 10 which comprises flanges 12 with threaded sections 14 ending in the area of receiver 10 below the area occupied by top of rod 30. Line A-A depicts the location of the area defined by the top of rod 30. Spinal implant 1 further comprises fastener portion 50 including head 52 and anchoring element in the form of polyaxial hook 58. Securing member has corresponding threads 22 to engage threads 14 of receiver 10 and to secure rod 30 within receiver 10. When securing member 20 is in its locked position, it applies force upon rod 30 which directly contacts and applies force upon head 52 forcing head 52 against the lower end of receiver 10 for preventing further pivotal and rotational movement of fastener 50 and receiver 10 relative to one another.

FIG. 8B shows spinal implant 1 having receiver 10 which comprises flanges 12 with threaded sections 14 ending in the area of receiver 10 below the area occupied by top of rod 30. Line A-A depicts the location of the area defined by the top of rod 30. Spinal implant 1 further comprises intermediate member 40 and fastener portion 50 including head 52 and anchoring element in the form of polyaxial hook 58. Securing member 20 has corresponding threads 22 to engage threads 14 of receiver 10 and to secure rod 30 within receiver 10. When securing member 20 is in its locked position, it applies force upon rod 30 which directly contacts and applies force upon intermediate member 40 and indirectly applies force on head 52 forcing head 52 against the lower end of receiver 10 for preventing further pivotal and rotational movement of fastener 50 and receiver 10 relative to one another.

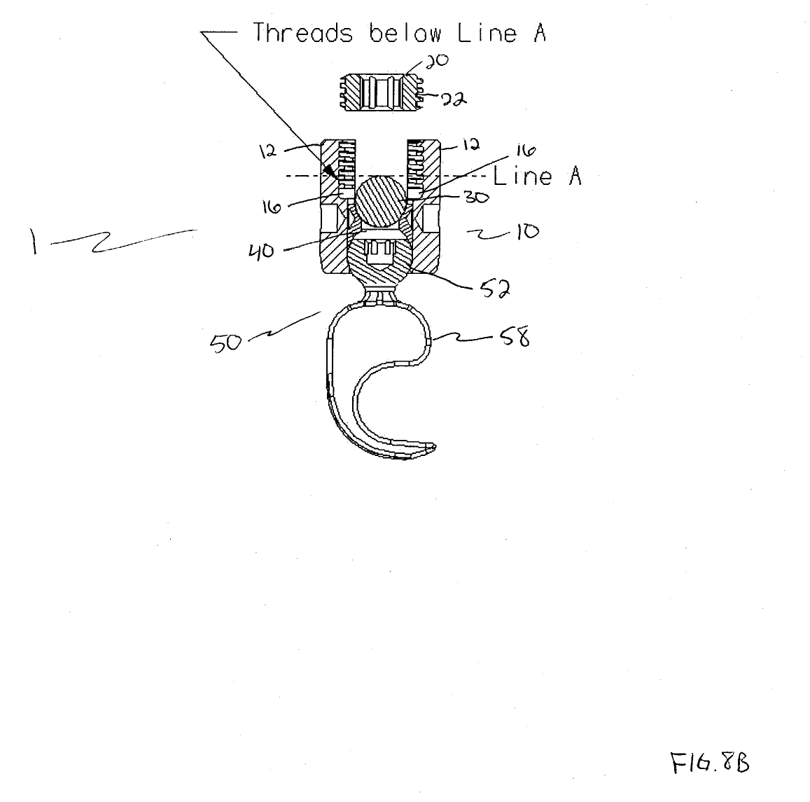

FIG. 9 depicts another embodiment of spinal implant 1. This embodiment represents a monoaxial screw having receiver 10 having flanges 12 with threaded sections 14 ending in the area of receiver 10 below the area occupied by top of rod 30. Line A-A depicts the location of the area defined by the top of rod 30. Spinal implant 1 further comprises fastener portion 50 in the form of monoaxial screw 56. Securing member 20 has corresponding threads 22 to engage threads 14 of receiver 10 to apply a force upon rod 30 positioned within receiver 10 and rod 30 contacts and applies a force upon the lower end of receiver 10 for preventing movement of rod 30 and receiver 10 relative to each other.

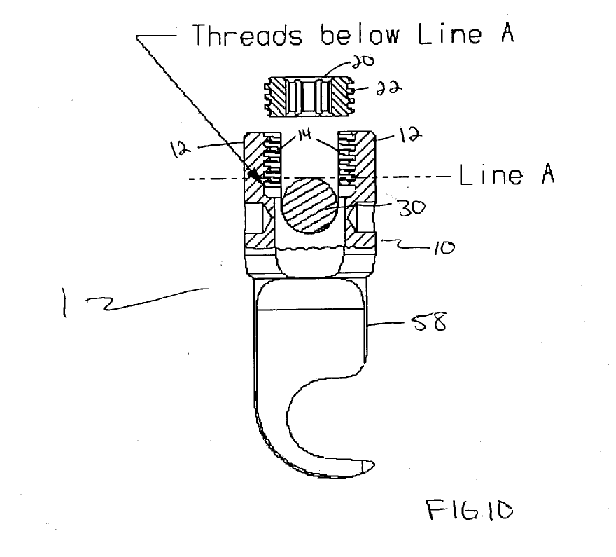

FIG. 10 represents another embodiment of spinal implant 1. This embodiment represents a monoaxial hook comprising receiver 10 having flanges 12 with threaded sections 14 ending in the area of receiver 10 below the area occupied by top of rod 30. Line A-A depicts the location of the area defined by the top of rod 30. Spinal implant 1 further comprises fastener portion 50 in the form of hook 58. Securing member 20 has corresponding threads 22 to engage threads 14 of receiver 10 to apply a force upon rod 30 positioned within receiver 10 and rod 30 contacts and applies a force upon the lower end of receiver 10 for preventing movement of rod 30 and receiver 10 relative to each other.

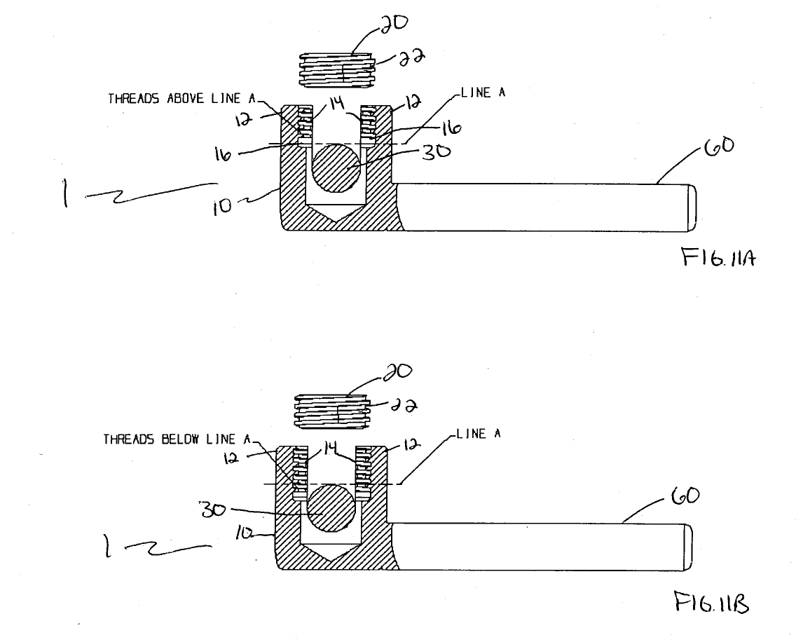

FIG. 11A represents a different style spinal implant 1. This implant is a sacral extender comprising receiver 10 having flanges 12 with threaded sections 14 ending above rod 30 and extender portion 60. Securing member 20 has corresponding threads 22 to engage threads 14 of receiver 10 to apply a force upon rod 30 positioned within receiver 10 and rod 30 contacts and applies a force upon the lower end of receiver 10 for preventing movement of rod 30 and receiver 10 relative to each other. Section 16 of receiver 10 does not contain any threads 14. Line A-A shows threads 14 ending above the top of rod 30.

FIG. 11B shows another embodiment of the different style spinal implant 1. Spinal implant 1 is a sacral extender comprising receiver 10 having flanges 12 with threaded sections 14 ending in the area of receiver 10 below the area occupied by top of rod 30. Line A-A depicts the location of the area defined by the top of rod 30. Spinal implant 1 further comprises extender portion 60. Securing member 20 has corresponding threads 22 to engage threads 14 of receiver 10 to apply a force upon rod 30 positioned within receiver 10 and rod 30 contacts and applies a force upon the lower end of receiver 10 for preventing movement of rod 30 and receiver 10 relative to each other.

It should be understood that the foregoing disclosure and description of the present invention are illustrative and explanatory thereof and various changes in the size, shape and materials as well as in the description of the preferred embodiment may be made without departing from the spirit of the invention. For example, intermediate member 40 may also be in the forms of collets, washers, compression caps or annular rings.

What is claimed is:

1. A bone anchor assembly comprising:
a. a bone fastener having an upper end and a lower end, a head at the upper end, and an anchoring element between the upper and lower ends;
b. a U-shaped receiver having an upper end and a lower end and having two flanges at its upper end, said receiver adapted to receive a stabilizing rod, a bore extending through the lower end of said receiver for receiving said fastener, threading beginning at said upper end and ending above the area of said receiver not occupied by said rod when said rod is in a locked position; and
c. a securing member having threads to engage the threading of said receiver to apply a force upon said rod positioned in said receiver and wherein said rod directly or indirectly contacts and applies a force upon said head for forcing said head against said lower end of the receiver for preventing further pivotal and rotational movement of said fastener and said receiver relative to one another.

2. The assembly as claimed in claim 1, wherein said fastener is a screw fastener, and wherein the anchoring element comprises screw threads extending between the upper and lower ends thereof.

3. The assembly as claimed in claim 1, wherein the anchoring element is a hook.

4. The assembly of claim 1, wherein the number of full width threads of the threading on each of the U-shaped member flanges are equal or substantially equal.

5. The assembly of claim 1, wherein the gap between the minor diameter of the securing member and the minor diameter of the receiver are minimized.

6. The assembly of claim 4, wherein in the gap between the minor diameter of the securing member and the minor diameter of the receiver are minimized.

7. The assembly of claim 1 wherein the thread designs are selected from the group consisting of square, metric, English, buttress and reverse angle thread designs.

8. The assembly of claim 7, wherein the thread design is square.

9. The assembly of claim 7 wherein the thread design is reverse angle.

10. The assembly of claim 1 wherein the rod directly contacts the head of the fastener.

11. The assembly of claim 2 wherein the rod indirectly contacts the head of the fastener.

12. A bone screw comprising:
a. a bone fastening end having threads to engage a bone; and
b. a U-shaped receiver end, said receiver having an upper end and a lower end and having two flanges at its upper end, said receiver adapted to receive a stabilizing rod, threading beginning at said upper end and ending above the area of said receiver not occupied by said rod when said rod is in a locked position; and
c. a securing member having threads to engage the threading of said receiver to apply a force upon said rod positioned in said receiver and wherein said rod contacts and applies a force upon said lower end of the receiver for preventing movement of said rod and said receiver relative to one another.

13. The assembly of claim 12, wherein the number of full width threads of the threading on each of the U-shaped member flanges are equal or substantially equal.

14. The assembly of claim 12, wherein the gap between the minor diameter of the securing member and the minor diameter of the receiver are minimized.

15. The assembly of claim 13, wherein the gap between the minor diameter of the securing member and the minor diameter of the receiver are minimized.

16. The assembly of claim 12 wherein the thread designs are selected from the group consisting of square, metric, English, buttress and reverse angle thread designs.

17. The assembly of claim 16, wherein the thread design is square.

18. The assembly of claim 16 wherein the thread design is reverse angle.

19. A bone hook comprising:
a. a bone engaging end having a hook to engage a bone; and
b. a U-shaped receiver end, said receiver having an upper end and a lower end and having two flanges at its upper end, said receiver adapted to receive a stabilizing rod, threading beginning at said upper end and ending above the area of said receiver not occupied by said rod when said rod is in a locked position; and
c. a securing member having threads to engage the threading of said receiver to apply a force upon said rod positioned in said receiver and wherein said rod contacts and applies a force upon said lower end of the receiver for preventing movement of said rod and said receiver relative to one another.

20. The assembly of claim 19, wherein the number of full width threads of the threading on each of the U-shaped member flanges are equal or substantially equal.

21. The assembly of claim 19, wherein the gap between the minor diameter of the securing member and the minor diameter of the receiver are minimized.

22. The assembly of claim 20, wherein in gap between the minor diameter of the securing member and the minor diameter of the receiver are minimized.

23. The assembly of claim 19 wherein the thread designs are selected from the group consisting of square, metric, English, buttress and reverse angle thread designs.

24. The assembly of claim 23, wherein the thread design is square.

25. The assembly of claim 23 wherein the thread design is reverse angle.

26. A bone anchor assembly comprising:
a. a bone fastener having an upper end and a lower end, a head at the upper end, and an anchoring element between the upper and lower ends thereof;
b. a U-shaped receiver having an upper end and a lower end and having two flanges at its upper end, said receiver adapted to receive a stabilizing rod, a bore extending through the lower end of said receiver for receiving said fastener, threading beginning at said upper end and ending in the area of said receiver above or below the area occupied by the top of said rod when said rod is in a locked position;
c. a securing member having threads to engage the threading of said receiver to apply a force upon said rod positioned in said receiver and wherein said rod directly or indirectly contacts and applies a force upon said head for forcing said head against said lower end of the receiver for preventing further pivotal and rotational movement of said fastener and said receiver relative to one another; and
d. wherein the number of full width threads of the threading on each of the U-shaped member flanges are equal or substantially equal.

27. The assembly as claimed in claim 26, wherein the gap between the minor diameter of the securing member and the minor diameter of the receiver are minimized.

28. The assembly as claimed in claim 26 or 27, wherein said fastener is a screw fastener, and wherein the anchoring element comprises screw threads extending between the upper and lower ends thereof.

29. The assembly as claimed in claim 26 or 27, wherein the anchoring element is a hook.

30. The assembly of claim 26 or 27 wherein the thread designs are selected from the group consisting of square, metric, English, buttress and reverse angle thread designs.

31. The assembly of claim 30, wherein the thread design is square.

32. The assembly of claim 30, wherein the thread design is reverse angle.

33. The assembly of claim 26 wherein the rod directly contacts the head of the fastener.

34. The assembly of claim 26 wherein the rod indirectly contacts the head of the fastener.

35. A bone screw comprising:
a. a bone fastening end having threads to engage a bone; and
b. a U-shaped receiver end, said receiver having an upper end and a lower end and having two flanges at its upper end, said receiver adapted to receive a stabilizing rod, threading beginning at said upper end and ending either above or below the area of said receiver not occupied by said rod when said rod is in a locked position;
c. a securing member having threads to engage the threading of said receiver to apply a force upon said rod positioned in said receiver and wherein said rod contacts and applies a force upon said lower end of the receiver for preventing movement of said rod and said receiver relative to one another; and
d. wherein the number of full width threads on the threading on each of the U-shaped flanges are equal or substantially equal.

36. The assembly of claim 35, wherein the gap between the minor diameter of the securing member and the minor diameter of the receiver are minimized.

37. The assembly of claim 35 or 36 wherein the thread designs are selected from the group consisting of square, metric, English, buttress and reverse angle thread designs.

38. The assembly of claim 37, wherein the thread design is square.

39. The assembly of claim 37 wherein the thread design is reverse angle.

40. A bone hook comprising:
a. a bone engaging end having a hook to engage a bone; and
b. a U-shaped receiver end, said receiver having an upper end and a lower end and having two flanges at its upper end, said receiver adapted to receive a stabilizing rod, threading beginning at said upper end and ending above or below the area of said receiver not occupied by said rod when said rod is in a locked position;
c. a securing member having threads to engage the threading of said receiver to apply a force upon said rod positioned in said receiver and wherein said rod contacts and applies a force upon said lower end of the receiver for preventing movement of said rod and said receiver relative to one another; and
d. wherein the number of full width threads of the threading on each of the U-shaped member flanges are equal or substantially equal.

41. The assembly of claim 40 wherein the gap between the minor diameter of the securing member and the minor diameter of the receiver are minimized.

42. The assembly of claim 40 or 41 wherein the thread designs are selected from the group consisting of square, metric, English, buttress and reverse angle thread designs.

43. The assembly of claim 42, wherein the thread design is square.

44. The assembly of claim 42 wherein the thread design is reverse angle.

45. A spinal implant comprising:
a. a U-shaped receiver end, said receiver having an upper end and a lower end and having two flanges at its upper end, said receiver adapted to receive a stabilizing rod, threading beginning at said upper end and ending above the area of said receiver not occupied by said rod when said rod is in a locked position;
b. a securing member having threads to engage the threading of said receiver to apply a force upon said rod positioned in said receiver and wherein said rod directly or indirectly contacts and applies a force upon said lower end of the receiver for preventing movement of said rod and said receiver relative to one another;
c. wherein the number of full width threads of the threading on each of the U-shaped member flanges are equal or substantially equal; and
d. wherein the spinal implant is selected from the group consisting of polyaxial and monoaxial types of screws, hooks, extenders, and connectors.

46. The spinal implant of claim 45 wherein the gap between the minor diameter of the securing member and the minor diameter of the receiver are minimized.

47. The spinal implant of claim 45 or 46 wherein the thread designs are selected from the group consisting of square, metric, English, buttress and reverse angle thread designs.

48. The spinal implant of claim 47, wherein the thread design is square.

49. The assembly of claim 47 wherein the thread design is reverse angle.
